(19) United States
(12) Patent Application Publication
(10) Pub. No.: US 2003/0208019 A1
(43) Pub. Date: Nov. 6, 2003

(54) Plastic lens composition, plastic lens, and process for producing the plastic lens

(76) Inventors: Kazuhiko Ooga, Oita-shi (JP); Tsuneo Tajima, Oita-shi (JP); Kai Kazufumi, Oita-shi (JP); Hiroshi Uchida, Oita-shi (JP)

Correspondence Address: SUGHRUE MION, PLLC, 2100 PENNSYLVANIA AVENUE, N.W., WASHINGTON, DC, 20037, US

(21) Appl. No.: 10/088,051

(22) PCT Filed: Feb. 26, 2002
(86) PCT No.: PCT/JP02/01758

(51) Int. Cl. C08F 220/70; A61F 2/16

(52) U.S. Cl. 526/307.6; 623/6.11; 526/307.4; 526/307.8; 526/313; 526/318.1; 526/318.2; 526/318.3; 526/318.44; 526/321; 526/326

(57) ABSTRACT

A composition for plastic lenses having a viscosity suitable for application to plastic lens materials and other optical materials and capable of giving a cured material having a relatively high refractive index and a low specific gravity, a plastic lens obtained by curing the composition, and a process for producing the plastic lens. A specific dicarboxylic acid component and a specific diol component are employed and the ratio of the components is adjusted to provide the composition for plastic lenses.

### CROSS-REFERENCE TO RELATED APPLICATION

This application is an application filed under 35 U.S.C. §111(a) claiming benefit, pursuant to 35 U.S.C. §119(e)(1) of the filing date of the Provisional Application 60/275,521 filed Mar. 8, 2001, pursuant to 35 U.S.C. §111(b).

### TECHNICAL FIELD

The present invention relates to a composition for plastic lenses, a plastic lens obtained by curing the composition and a process for producing the plastic lens.

More specifically, the present invention relates to a composition for plastic lenses, having a viscosity suitable for the application to plastic lens materials and other optical materials and capable of providing a cured material having a relatively high refractive index and a low specific gravity, and also relates to a plastic lens obtained by curing the composition and a process for producing the plastic lens.

### BACKGROUND ART

Organic glasses are lightweight as compared with inorganic glasses and therefore, organic glasses comprising a polymer of diethylene glycol bis(allyl carbonate), methyl methacrylate or the like, represented by CR-39 (trade name, produced by PPG), have been heretofore used. However, these organic glasses are disadvantageous in that the refractive index, which is from 1.49 to 1.50, is relatively low as compared with inorganic glasses (refractive index of white crown glass: 1.523), the thickness is greater than inorganic glasses, canceling the effect of reducing the weight, and when used as a lens for visual acuity correction, the higher degree of myopia gives worse looking.

In order to solve these problems, various organic glasses using a diallyl phthalate-based monomer have been proposed. However, these are fragile or have problems in transmittance. If this monomer is diluted with a monofunctional polymerizable monomer so as to improve these properties, its resistance to heat or solvents is impaired, resulting in insufficient capability as an organic glass.

An allyl ester having an allyl ester group at the terminal and having inside thereof the following structure derived from a polyvalent saturated carboxylic acid and a polyhydric saturated alcohol is also known.

CH₂═CHCH₂O{CORCOOB′O}ₙCORCOOCH₂CH═CH₂

wherein R represents a divalent organic residue having from 1 to 20 carbon atoms, B′ represents a divalent organic residue derived from a diol, and n is a number from 1 to 20.

These allyl esters provide a cured material having excellent impact resistance. However, since an aliphatic hydrocarbon B′ is used inside, even if terephthalic acid or isophthalic acid is used as the polyvalent saturated carboxylic acid, the refractive index is disadvantageously lower than that of the cured material of a diallyl terephthalate monomer or a diallyl isophthalate monomer itself.

The present inventors have proposed a composition for plastic lenses, containing an organic residue derived from a compound having an aromatic ring and two or more hydroxyl groups, in Japanese Unexamined Patent Publications No. 3-199218 (JP-A-3-199218), No. 3-258820 (JP-A-3-258820), No. 7-33830 (JP-A-7-33830) and No. 7-33834 (JP-A-7-33834).

Japanese Unexamined Patent Publication No. 7-138334 (JP-A-7-138334) also proposes a composition for plastic lenses, containing an organic residue derived from the compound having an aromatic ring and two or more hydroxyl groups. Those compositions proposed are, however, not necessarily optimal from the standpoint of producing a composition having low viscosity and forming the cured material into a plastic lens having high refractive index.

Furthermore, the compositions of JP-A-7-138334 cannot achieve low viscosity unless the amount of the compound having an aromatic ring and two or more hydroxyl groups used is greatly reduced or the reactive monomer is in a large amount. However, if the amount of the compound having an aromatic ring and two or more hydroxyl groups used is drastically reduced, a high refractive index of 1.58 or more cannot be obtained. Also, if the reactive monomer is used in a large amount, the heat resistance disadvantageously deteriorates.

### DISCLOSURE OF INVENTION

In order to solve the above-described problems, the object of the present invention is to provide a composition for plastic lenses, having a viscosity suitable for application to plastic lens materials and other optical materials and capable of providing a cured material having a relatively high refractive index and low specific gravity, as well as a plastic lens obtained by curing the composition and a process for producing the plastic lens.

As a result of extensive investigations to solve the above-described problems, the present inventors have found that by use of a dicarboxylic acid component having a specific structure and a diol component and adjusting the molar ratio therebetween, there can be provided a composition for plastic lenses, having a viscosity suitable for application to plastic lens materials and other optical materials and having a relatively high refractive index and low specific gravity. The present invention has been accomplished based on this finding.

More specifically, the present invention (I) provides a composition for plastic lenses, comprising the following component (α) and component (β) as essential components:

Component (α):

at least one compound selected from the compounds having at least one group represented by the following formula (1) as a terminal group and having a group represented by the following formula (2) as a repeating unit;

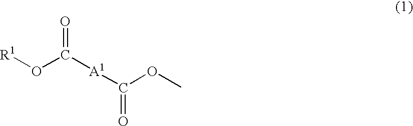

wherein each R¹ independently represents an allyl group or a methallyl group and each A¹ independently represents an organic residue derived from a divalent carboxylic acid or carboxylic anhydride;

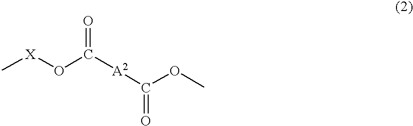

wherein each A² independently represents an organic residue derived from a divalent carboxylic acid or carboxylic anhydride and each X independently represents an organic residue and the X's represent one or more organic residues essentially containing an organic residue derived from a compound having an aromatic ring and two or more hydroxyl groups, provided that by the ester bonding, X can have a branched structure having a group represented by formula (1) as a terminal group and a group represented by formula (2) as a repeating unit;

Component (β):

at least one compound selected from the compounds represented by the following formula (3):

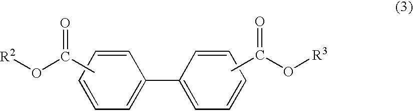

wherein R² and R³ each independently represents an allyl group or a methallyl group.

The present invention (II) provides a composition for plastic lenses, comprising the following component (α), component (β) and component (γ) as essential components:

Component (α):

at least one compound selected from the group consisting of the compounds having at least one group represented by the following formula (1) as a terminal group and having a group represented by the following formula (2) as a repeating unit;

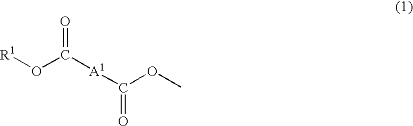

wherein each R¹ independently represents an allyl group or a methallyl group and each A¹ independently represents an organic residue derived from a divalent carboxylic acid or a carboxylic anhydride;

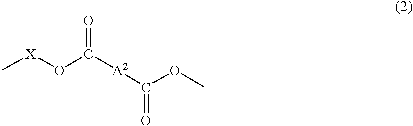

wherein each A² independently represents an organic residue derived from a divalent carboxylic acid or carboxylic anhydride and each X is independently an organic residue and the X's represent one or more organic residues essentially containing an organic residue derived from a compound having an aromatic ring and two or more hydroxyl groups, provided that by the ester bonding, X can have a branched structure having a group represented by formula (1) as a terminal group and a group represented by formula (2) as a repeating unit;

Component (β):

at least one compound selected from the group consisting of the compounds represented by the following formula (3);

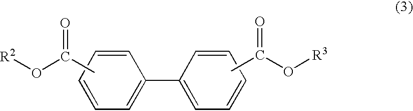

wherein R² and R³ each independently represents an allyl group or a methallyl group;

Component (γ):

at least one monofunctional compound selected from the group consisting of monofunctional compounds having two or more benzene rings within one molecule, monofunctional compounds having a naphthalene ring within one molecule and monofunctional compounds having a benzene ring and a halogen atom within one molecule.

The present invention (III) provides the composition for plastic lenses described in either one of the present invention (I) and the present invention (II), wherein at least one radical polymerization initiator is contained in an amount of 0.1 to 10 parts by mass per 100 parts by mass of whole curable components in the composition for plastic lenses.

The present invention (IV) provides a plastic lens obtained by curing the composition for plastic lenses described in any one of the present invention (I) to the present invention (III).

The present invention (V) provides a process for producing the plastic lens of the present invention (IV).

### BRIEF DESCRIPTION OF DRAWINGS

FIG. 1 is a 400 MHz ¹H-NMR spectrum chart of the allyl ester compound produced in Production Example 1.

FIG. 2 is an FT-IR spectrum chart of the allyl ester compound produced in Production Example 1.

### BEST MODE FOR CARRYING OUT THE INVENTION

The present invention is described in detail below. The compositions for plastic lenses of the present invention (I) and the present invention (II) will now be described.

The present invention (I) provides a composition for plastic lenses, comprising the following component (α) and the following component (β) as essential components:

Component (α):

at least one compound selected from the group consisting of the compounds having at least one group represented by formula (1) above as a terminal group and having a group represented by formula (2) above as a repeating unit;

Component (β):

at least one compound selected from the group consisting of the compounds represented by formula (3) above:

The present invention (II) provides a composition for plastic lenses, comprising the following component (α), component (β) and component (γ) as essential components:

Component (α):

at least one compound selected from the group consisting of the compounds having at least one group represented by formula (1) above as a terminal group and having a group represented by formula (2) above as a repeating unit;

Component (β):

at least one compound selected from the group consisting of the compounds represented by formula (3) above;

Component (γ):

at least one monofunctional compound selected from the group consisting of a monofunctional compound having two or more benzene rings within one molecule, a monofunctional compound having a naphthalene ring within one molecule and a monofunctional compound having a benzene ring and a halogen atom within one molecule.

The term “whole curable components” as used herein refers to the total amount of radical polymerizable components contained in the composition for plastic lenses of the present invention.

Component (α):

At least one compound selected from the group consisting of the compounds having at least one group represented by formula (1) as a terminal group and a group represented by formula (2) as a repeating unit, which is an essential component of the present invention (I) or the present invention (II), is described below (hereinafter referred to as “component (α)”).

In formula (1), each R¹ independently represents an allyl group or a methallyl group. In formula (1), each A¹ independently represents an organic residue derived from a divalent carboxylic acid or carboxylic anhydride. In formula (2), each A² independently represents an organic residue derived from a divalent carboxylic acid or carboxylic anhydride. Furthermore, in formula (2), each X independently represents an organic residue and the X's represent one or more organic residues essentially containing an organic residue derived from a compound having an aromatic ring and two or more hydroxyl groups.

The term “each R¹ independently represents an allyl group or a methallyl group” as used herein means that the moieties represented by R¹ in the terminal group represented by formula (1) as an essential component of the composition for plastic lenses of the present invention may all be an allyl group or a methallyl group or may be partially an allyl group with the remaining being a methallyl group.

A¹ in formula (1) and A² in formula (2) each represent an organic residue derived from a divalent carboxylic acid or carboxylic anhydride. Examples of the “divalent carboxylic acid or carboxylic anhydride” as used herein include the following compounds. However, the present invention is of course not limited to these specific examples.

Examples include aliphatic dicarboxylic acids and aliphatic dicarboxylic anhydrides, such as succinic acid, succinic anhydride, glutaric acid, glutaric anhydride, adipic acid, malonic acid, malonic anhydride, 2-methylsuccinic acid and 2-methylsuccinic anhydride; dicarboxylic acids having an alicyclic structure and dicarboxylic anhydrides having an alicyclic structure, such as 1,4-cyclohexanedicarboxylic acid, 1,3-cyclohexanedicarboxylic acid, 1,2-cyclohexanedicarboxylic acid, 1,2-cyclohexanedicarboxylic anhydride, 4-methylcyclohexane-1,2-dicarboxylic acid and 4-methylcyclohexane-1,2-dicarboxylic anhydride; and aromatic dicarboxylic acids and aromatic dicarboxylic anhydrides, such as terephthalic acid, isophthalic acid, phthalic acid, phthalic anhydride, biphenyl-2,2′-dicarboxylic acid, biphenyl-2,2′-dicarboxylic anhydride, biphenyl-3,3′-dicarboxylic acid and biphenyl-4,4′-dicarboxylic acid.

Among these, from the standpoint of the compound maintaining a high refractive index, preferred are aromatic dicarboxylic acids and aromatic dicarboxylic anhydrides, such as terephthalic acid, isophthalic acid, phthalic acid, phthalic anhydride, biphenyl-2,2′-dicarboxylic acid, biphenyl-2,2′-dicarboxylic anhydride, biphenyl-3,3′-dicarboxylic acid and biphenyl-4,4′-dicarboxylic acid, more preferred are isophthalic acid, biphenyl-2,2′-dicarboxylic acid and biphenyl-2,2′-dicarboxylic anhydride.

The term “each A¹ independently represents an organic residue derived from a divalent carboxylic acid or carboxylic anhydride,” or “each A² independently represents an organic residue derived from a divalent carboxylic acid or carboxylic anhydride” as used herein means that the moieties represented by A¹ in the terminal group represented by formula (1) in the component (α) as an essential component of the composition for plastic lenses of the present invention, or the moieties represented by A² in the repeating unit represented by formula (2) in the component (α) as an essential component of the composition for plastic lenses of the present invention (hereinafter “A¹” and “A²” are collectively referred to as “A”), may all be organic residues derived from divalent carboxylic acids or carboxylic anhydrides having the same structure, all may be organic residues derived from divalent carboxylic acids or carboxylic anhydrides having different structures, or may partially be organic residues derived from divalent carboxylic acids or carboxylic anhydrides having the same structure with the remaining being organic residues derived from divalent carboxylic acids or carboxylic anhydrides having different structures.

More specifically, in the following formula (4) which is one example of the component (α) as an essential component of the composition of plastic lenses of the present invention, A's in the number of k contained in the structure are independent of each other:

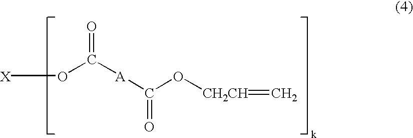

wherein each A independently represents an organic residue derived from a divalent carboxylic acid or carboxylic anhydride, k is an integer of 2 or more, and X represents an organic residue derived from a compound having an aromatic ring and two or more hydroxyl groups).

In formula (4), for example, A's in the number of k may all be organic residues derived from divalent carboxylic acids or carboxylic anhydrides having different structures (that is, one organic residue is derived from individual divalent carboxylic acids or carboxylic anhydrides having k kinds of structures) or may all be organic residues derived from divalent carboxylic acids or carboxylic anhydrides having the same structure (that is, organic residues in the number of k are derived from divalent carboxylic acids or carboxylic anhydrides having one kind of structure). A mixed structure where some of A's in the number of k are organic residues derived from divalent carboxylic acids or carboxylic anhydrides having the same structure and some others are organic residues derived from divalent carboxylic acids or carboxylic anhydrides having different structures, may also be used.

The term “each X independently represents an organic residue” as used herein means that in the following formula (5) as one example of the repeating unit represented by formula (2), the X's in the number of m contained in the repeating structure are organic residues independent of each other:

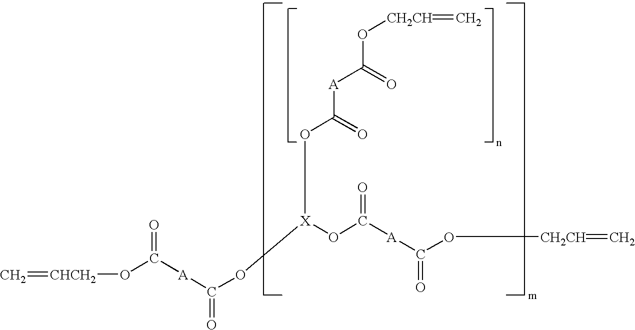

wherein each X independently represents an organic residue and the X's represent one or more organic residues essentially containing an organic residue derived from a compound having an aromatic ring and two or more hydroxyl groups, m is 0 or an integer of 1 or more, n is 0 or an integer of 1 or more, and each A independently represents an organic residue derived from a divalent carboxylic acid or carboxylic anhydride).

For example, in formula (5), the X's in the number of m may all be organic residues derived from different compounds having an aromatic ring and two or more hydroxyl groups (that is, one organic residue is derived from individual compounds of m kinds having an aromatic ring and two or more hydroxyl groups) or all may be organic residues derived from the same compound (that is, organic residues in the number of m are derived from one kind of compound having an aromatic ring and two or more hydroxyl groups). A mixed structure where some of X's in the number of m are organic residues derived from the same compound and some others are organic residues derived from different kinds of compounds, may also be used. Moreover, in this mixed structure, the whole may be completely random or a part may be repeated.

The term “one or more organic residues essentially containing an organic residue derived from a compound having an aromatic ring and two or more hydroxyl groups” as used herein means that, in formula (5) as one example of the repeating unit represented by formula (2), a part or all of the X's in the number of m contained in the repeating structures contain an organic residue derived from a compound having an aromatic ring and two or more hydroxyl groups.

For example, in formula (5), the X's in the number of m may all be an organic residue derived from a compound containing an aromatic ring (that is, organic residues in the number of m are derived from at least one compound having an aromatic ring and two or more hydroxyl groups) or may have a mixed structure where some of the X's in the number of m are an organic residue derived from a compound having an aromatic ring and two or more hydroxyl groups and some others are an organic residue derived from another kind of compound. Furthermore, in the mixed structure, the whole may be completely random or a part may be repeated.

By the ester bonding, X can have a branched structure containing formula (1) as a terminal group and formula (2) as a repeating unit. More specifically, for example, when an organic residue derived from 1,3,5-tris(2-hydroxyethyl)benzene as one example of the compound having an aromatic ring and three or more hydroxyl groups is present in X, the component (α) as an essential component of the composition for plastic lenses of the present invention (I) or (II) can have a partial structure represented by the following formula (6):

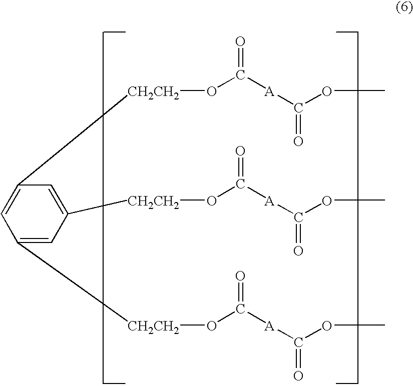

Each A independently represents an organic residue derived from a divalent carboxylic acid or carboxylic anhydride.

In formula (2), each X is independently an organic residue and the X's represent one or more organic residues essentially containing an organic residue derived from a compound having an aromatic ring and two or more hydroxyl groups. Examples of the “compound having an aromatic ring and two or more hydroxyl groups” as used herein include the compounds having an aromatic ring and two or more hydroxyl groups, represented by the following formulae (7) to (9). Needless to say, however, the present invention is not limited to these specific examples.

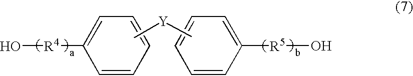

wherein each R⁴ independently represents an organic group selected from the organic groups represented by the following structural formulae (10) to (12), each R⁵ independently represents an organic group selected from the organic groups represented by the following structural formulae (13) to (15), a and b are each independently 0 or an integer of 1 to 10, and Y represents an organic group represented by the following structural formulae (16) or (17);

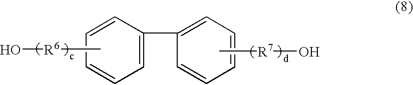

wherein each R⁶ independently represents an organic group selected from the organic groups represented by structural formulae (10) to (12), each R⁷ independently represents an organic group selected from the organic groups represented by structural formulae (13) to (15), and c and d are each independently 0 or an integer of 1 to 10;

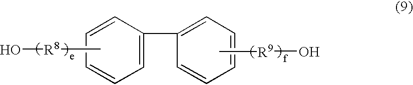

wherein each R⁸ independently represents a methylene group or an organic group selected from the organic groups represented by structural formulae (10) to (12), each R⁹ represents an organic group selected from the organic groups represented by structural formulae (13) to (15), and e and f are each independently 0 or an integer of 1 to 10.

—CH₂Ch₂O— (10) 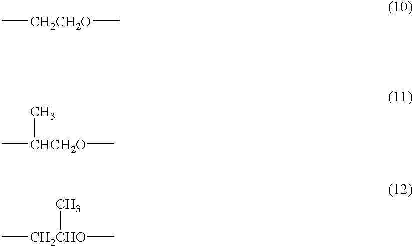
—OCH₂CH₂— (13) 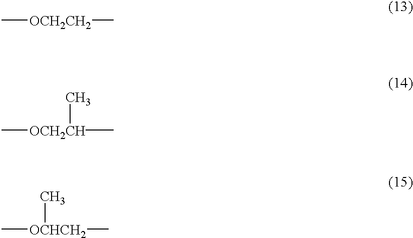 —CH₂— (16)

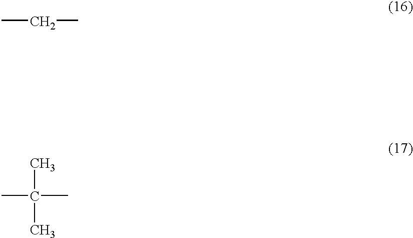

In formula (7); the R⁴s in the number of a may all be organic groups having the same structure, may all be organic groups having different structures, or may be partially organic groups having the same structure with the remaining being organic groups having different structures, where, however, R⁴ must be selected from the organic groups represented by structural formulae (10) to (12).

In formula (7), the R⁵s in the number of b may all be organic groups having the same structure, may all be organic groups having different structures or may be partially organic groups having the same structure with the remaining being organic groups having different structures, where, however, R⁵ must be selected from the organic groups represented by structural formulae (13) to (15).

In formula (7), a and b are each independently 0 or an integer of 1 to 10.

In formula (7), Y represents any one organic group selected from those of structural formulae (16) and (17).

Specific examples of the compound having an aromatic ring and two or more hydroxyl groups, represented by formula (7), include bisphenol F, 2 mol ethylene oxide adduct of bisphenol F, 4 mol ethylene oxide adduct of bisphenol F, 6 mol ethylene oxide adduct of bisphenol F, 8 mol ethylene oxide adduct of bisphenol F, 2 mol propylene oxide adduct of bisphenol F, 4 mol propylene oxide adduct of bisphenol F, 6 mol propylene oxide adduct of bisphenol F, 8 mol propylene oxide adduct of bisphenol F, bisphenol A, 2 mol ethylene oxide adduct of bisphenol A, 4 mol ethylene oxide adduct of bisphenol A, 6 mol ethylene oxide adduct of bisphenol A, 8 mol ethylene oxide adduct of bisphenol A, 2 mol propylene oxide adduct of bisphenol A, 4 mol propylene oxide adduct of bisphenol A, 6 mol propylene oxide adduct of bisphenol A, and 8 mol propylene oxide adduct of bisphenol A. Needless to say, however, the present invention is not limited to these specific examples.

Among these compounds, in view of easy availability of starting materials and reactivity, preferred are 2 mol ethylene oxide adduct of bisphenol A, 3 mol ethylene oxide adduct of bisphenol A, 4 mol ethylene oxide adduct of bisphenol A, 6 mol ethylene oxide adduct of bisphenol A, 8 mol ethylene oxide adduct of bisphenol A, 2 mol propylene oxide adduct of bisphenol A, 4 mol propylene oxide adduct of bisphenol A, 6 mol propylene oxide adduct of bisphenol A, 8 mol propylene oxide adduct of bisphenol A, 2 mol ethylene oxide adduct of bisphenol F, 3 mol ethylene oxide adduct of bisphenol F and 4 mol ethylene oxide adduct of bisphenol F, more preferred are 2 mol ethylene oxide adduct of bisphenol A, 2 mol propylene oxide adduct of bisphenol A, 2 mol ethylene oxide adduct of bisphenol F, 3 mol ethylene oxide adduct of bisphenol F and 4 mol ethylene oxide adduct of bisphenol F.

In formula (8), the R⁶s in the number of c may all be organic groups having the same structure, may all be organic groups having different structures, or may be partially organic groups having the same structure with the remaining being organic groups having different structures, where, however, R⁶ must be selected from the organic groups represented by structural formulae (10) to (12).

In formula (8), the R⁷s in the number of d may all be organic groups having the same structure, may all be organic groups having different structures or may be partially organic groups having the same structure with the remaining being organic groups having different structures, where, however, R⁷ must be selected from the organic groups represented by structural formulae (13) to (15).

In formula (8), c and d are each independently 0 or an integer of 1 to 10.

Specific examples of the compound having an aromatic ring and two or more hydroxyl groups, represented by formula (8), include 4,4′-dihydroxybiphenyl, 2 mol ethylene oxide adduct of 4,4′-dihydroxybiphenyl, 4 mol ethylene oxide adduct of 4,4′-dihydroxybiphenyl, 6 mol ethylene oxide adduct of 4,4′-dihydroxybiphenyl, 8 mol ethylene oxide adduct of 4,4′-dihydroxybiphenyl, 2 mol propylene oxide adduct of 4,4′-dihydroxybiphenyl, 4 mol propylene oxide adduct of 4,4′-dihydroxybiphenyl, 6 mol propylene oxide adduct of 4,4′-dihydroxybiphenyl, 8 mol propylene oxide adduct of 4,4′-dihydroxybiphenyl, 2,2′-dihydroxybiphenyl, 2 mol ethylene oxide adduct of 2,2′-dihydroxybiphenyl, 4 mol ethylene oxide adduct of 2,2′-dihydroxybiphenyl, 6 mol ethylene oxide adduct of 2,2′-dihydroxybiphenyl, 8 mol ethylene oxide adduct of 2,2′-dihydroxybiphenyl, 2 mol propylene oxide adduct of 2,2′-dihydroxybiphenyl, 4 mol propylene oxide adduct of 2,2′-dihydroxybiphenyl, 6 mol propylene oxide adduct of 2,2′-dihydroxybiphenyl and 8 mol propylene oxide adduct of 2,2′-dihydroxybiphenyl. Needless to say, however, the present invention is not limited to these specific examples.

Among these compounds, 2 mol ethylene oxide adduct of 2,2′-dihydroxybiphenyl, 2 mol ethylene oxide adduct of 4,4′-dihydroxybiphenyl and 2 mol propylene oxide adduct of 4,4′-dihydroxybiphenyl are preferred. More preferred is 2 mol ethylene oxide adduct of 2,2′-dihydroxybiphenyl.

In formula (9), the R⁸s in the number of e may all be organic groups having the same structure, may all be organic groups having different structures, or may be partially organic groups having the same structure with the remaining being organic groups having different structures, where, however, R⁸ must be selected from a methylene group and the organic groups represented by structural formulae (10) to (12).

In formula (9), the R⁹s in the number of f may all be organic groups having the same structure, may all be organic groups having different structures, or may be partially organic groups having the same structure with the remaining being organic groups having different structures, where, however, R⁹ must be selected from a methylene group and the organic groups represented by structural formulae (13) to (15).

In formula (9), e and f are each independently 0 or an integer of 1 to 10.

Specific examples of the compound having an aromatic ring and two or more of hydroxyl groups, represented by formula (9), include p-xylylene glycol, m-xylylene glycol, o-xylylene glycol, 1,4-bis(2-hydroxyethoxy)benzene, 1,3-bis(2-hydroxyethoxy)benzene, 1,2-bis(2-hydroxyethoxy)benzene, 4 mol ethylene oxide adduct of hydroquinone, 6 mol ethylene oxide adduct of hydroquinone, 8 mol ethylene oxide adduct of hydroquinone, 4 mol ethylene oxide adduct of catechol, 6 mol ethylene oxide adduct of catechol and 8 mol ethylene oxide adduct of catechol.

Among these compounds, p-xylylene glycol, m-xylylene glycol, o-xylylene glycol, 1,4-bis(2-hydroxyethoxy)benzene, 1,3-bis(2-hydroxyethoxy)benzene and 1,2-bis(2-hydroxy-ethoxy)benzene are preferred. More preferred are p-xylylene glycol, m-xylylene glycol and o-xylylene glycol.

In combination with the compound having an aromatic ring and two or more hydroxyl groups, another compound having a hydroxyl group can be used. Specific examples thereof include diethylene glycol, triethylene glycol, propylene glycol, 1,3-butanediol, 1,4-butanediol, 1,4-cyclohexanedimethanol, 1,3-cyclohexanedimethanol, 1,1-cyclohexanedimethanol and 2-methyl-1,1-cyclohexanedimethanol. Needless to say, however, the present invention is not limited to these specific examples.

The repeating number of the group represented by formula (2) which is a repeating unit of the component (α) as an essential component of the composition for plastic lenses of the present invention is not particularly limited. A mixture of materials having various repeating numbers may also be used. Furthermore, a compound having a repeating number of 0 (namely, the compound represented by the following formula (18)) and a compound having a repeating number of an integer of 1 or more may be used in combination. However, use of only a compound having a repeating number of 0 is disadvantageous in achieving the object of the present invention.

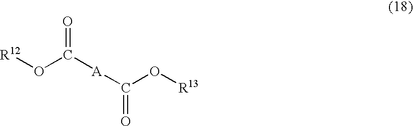

wherein A represents an organic residue derived from a divalent carboxylic acid or carboxylic anhydride, and R¹² and R¹³ each independently represents an allyl group or a methallyl group.

In this specification, the component (α) as an essential component of the composition for plastic lenses of the present invention is defined as not containing the remaining compound represented by formula (18).

More specifically, this means that when diallyl biphenyl-2,2′-dicarboxylate is used as a starting material in the production of the component (α) and the diallyl biphenyl-2,2′-dicarboxylate remains, the remaining diallyl biphenyl-2,2′-dicarboxylate is not contained in the component (α) but is contained in at least one compound selected from the compounds represented by formula (3).

In the case where diallyl succinate is used as a starting material in the production of the component (α) and the diallyl succinate remains, the remaining diallyl succinate is not contained in either the component (α) or at least one compound selected from the compounds represented by formula (3).

In formula (18), A represents an organic residue derived from a divalent carboxylic acid or carboxylic anhydride. Examples of the “divalent carboxylic acid or carboxylic anhydride” as used herein include the following compounds.

Examples thereof include aliphatic dicarboxylic acids and aliphatic dicarboxylic anhydrides, such as succinic acid, succinic anhydride, glutaric acid, glutaric anhydride, adipic acid, malonic acid, malonic anhydride, 2-methylsuccinic acid and 2-methylsuccinic anhydride; dicarboxylic acids having an alicyclic structure and dicarboxylic anhydrides having an alicyclic structure, such as 1,4-cyclohexanedicarboxylic acid, 1,3-cyclohexanedicarboxylic acid, 1,2-cyclohexanedicarboxylic acid, 1,2-cyclohexanedicarboxylic anhydride, 4-methylcyclohexane-1,2-dicarboxylic acid and 4-methylcyclohexane-1,2-dicarboxylic anhydride; and aromatic dicarboxylic acids and aromatic dicarboxylic anhydrides, such as terephthalic acid, isophthalic acid, phthalic acid, phthalic anhydride, biphenyl-2,2′-dicarboxylic acid, biphenyl-2,2′-dicarboxylic anhydride, biphenyl-3,3′-dicarboxylic acid and biphenyl-4,4′-dicarboxylic acid. However, the present invention is of course not limited to these specific examples.

Among these compounds, from the standpoint of maintaining a high refractive index of the compound, preferred are aromatic dicarboxylic acids and anhydrides thereof, such as terephthalic acid, isophthalic acid, phthalic acid, phthalic anhydride, biphenyl-2,2′-dicarboxylic acid, biphenyl-2,2′-dicarboxylic anhydride and biphenyl-3,3′-dicarboxylic acid, more preferred are isophthalic acid, biphenyl-2,2′-dicarboxylic acid and biphenyl-2,2′-dicarboxylic anhydride.

The repeating number of the group represented by formula (2) which is a repeating unit of the component (α) as an essential component of the composition for plastic lenses of the present invention is usually an integer of preferably 1 to 30. If a component (α) comprising only a compound having a repeating number in excess of 30 is used in the composition for plastic lenses, the allyl group concentration decreases and this may cause adverse effects, for example, in the process of curing, the curing may be retarded or a part of the compound may remain uncured to reduce the physical properties of the cured material, such as mechanical properties. In all compounds contained in the component (α), the repeating number is preferably an integer of 1 to 30, more preferably from 1 to 20, still more preferably from 1 to 10.

In the production of the component (α) as an essential component of the composition for plastic lenses of the present invention, the compound represented by formula (18) as a starting material remains depending on the production conditions but the component represented by formula (18) may be used as it is in the composition for plastic lenses without removing the compound. However, in the case of using the component in the composition for plastic lenses of the present invention (I) or (II), it may be disadvantageous to allow the compound represented by formula (18) to be present in excess of 95% by mass based on the entire curable components, because the impact resistance of the cured material excessively deteriorates.

The amount of the component (α) blended in the composition for plastic lenses of the present invention is preferably from 10 to 95% by mass, more preferably from 15 to 70% by mass, still more preferably from 20 to 60% by mass, based on whole curable components. If the amount of the component (α) blended in the composition for plastic lenses of the present invention is less than 10% by mass based on whole curable components, the cured material obtained by curing the composition for plastic lenses is difficult to maintain good impact resistance and this is not preferred. On the other hand, if the amount of the component (α) blended in the composition for plastic lenses of the present invention exceeds 95% by mass based on whole curable components, the composition is highly probably elevated in the viscosity to an extreme extent and this is not preferred.

Component (β):

At least one compound selected from the compounds represented by formula (3), which is an essential component of the present invention (I) or the present invention (II) is described below (hereinafter simply referred to as component (β)).

In the composition of the present invention, a component (β) may be used for the purpose of not reducing the refractive index of the cured material obtained from the composition of the present invention (I) or (II) to less than 1.58, adjusting the viscosity at 25° C. of the composition to 1,000 mPa·s or less and at the same time, maintaining the heat resistance.

Specific examples of the component (β) include diallyl biphenyl-2,2′-dicarboxylate, dimethallyl biphenyl-2,2′-dicarboxylate, allylmethallyl biphenyl-2,2′-dicarboxylate, diallyl biphenyl-3,3′-dicarboxylate, dimethallyl biphenyl-3,3′-dicarboxylate and allylmethallyl biphenyl-3,3′-dicarboxylate. Needless to say, however, the present invention is not limited to these specific examples.

The amount of the component (β) blended in the composition of the present invention based on whole curable components varies depending on the kind of the compound used but is preferably from 5 to 90% by mass, more preferably from 5 to 50% by mass, still more preferably from 5 to 30% by mass, based on whole curable components. If the amount of the component (β) blended is less than 5% by mass based on whole curable components, the viscosity of the composition excessively increases and this is not preferred. On the other hand, if the amount of the component (β) blended exceeds 90% by mass based on whole curable components, the cured material obtained by curing the composition for plastic lenses can hardly maintain the impact resistance and this is also not preferred.

Component (γ):

At least one monofunctional compound selected from the group consisting of a monofunctional compound having two or more benzene rings within one molecule, a monofunctional compound having a naphthalene ring within one molecule and a monofunctional compound having a benzene ring and a halogen atom within one molecule, which is an essential component of the present invention (II), is described below (hereinafter referred to as “component (γ)”).

In the present invention (II), a component (γ) is used.

The term “monofunctional compound” as used herein means a compound having only one radical polymerizable functional group within one molecule.

The component (γ) is used manly for reducing the viscosity of the cured material, maintaining or elevating the refractive index of the cured material and enhancing the impact resistance of the cured material.

Specific examples of the monofunctional compound having two or more benzene rings within one molecule as used herein include (meth)allyl p-phenylbenzoate, (meth)allyl m-phenylbenzoate, (meth)allyl o-phenylbenzoate, (meth)acryloyloxyethyl-4-phenylbenzoate, (meth)acryloyloxy-ethyl-3-phenylbenzoate, (meth)acryloyloxyethyl-2-phenyl-benzoate, diphenyl maleate, dibenzyl maleate, diphenyl fumarate and dibenzyl fumarate.

Among these compounds, preferred are (meth)allyl p-phenylbenzoate, (meth)allyl m-phenylbenzoate and (meth)allyl o-phenylbenzoate and on taking account of easy availability of the starting materials, most preferred is (meth)allyl p-phenylbenzoate.

Specific examples of the monofunctional compound having a naphthalene ring within one molecule as used herein include (meth)allyl α-naphthoate, (meth)allyl β-naphthoate, (meth)acryloyloxyethyl-α-naphthalene carboxylate and (meth)acryloyloxyethyl-β-naphthalene carboxylate. Needless to say, however, the present invention is not limited to these specific examples.

Among these compounds, in view of easy availability of the starting materials, allyl α-naphthoate and allyl β-naphthoate are preferred.

Specific examples of the monofunctional compound having a benzene ring and a halogen atom within one molecule as used herein include (meth)allyl o-chlorobenzoate, (meth)allyl m-chlorobenzoate, (meth)allyl p-chlorobenzoate, (meth)allyl 2,6-dichlorobenzoate, (meth)allyl 2,4-dichlorobenzoate, (meth)allyl 2,4,6-trichlorobenzoate, (meth)allyl o-bromobenzoate, (meth)allyl m-bromobenzoate, (meth)allyl p-bromobenzoate, (meth)allyl 2,6-dibromobenzoate, (meth)allyl 2,4-dibromobenzoate and (meth)allyl 2,4,6-tribromobenzoate. Needless to say, however, the present invention is not limited to these specific examples.

Among these compounds, in view of easy availability of starting materials, preferred are allyl o-chlorobenzoate, allyl m-chlorobenzoate, allyl p-chlorobenzoate, allyl 2,6-dichlorobenzoate and allyl 2,4-dichlorobenzoate.

The term “(meth)allyl” as used herein includes allyl and methallyl. The term “(meth)acryloyl” as used herein includes acryloyl and methacryloyl.

In the present invention, the amount of the component (γ) blended varies depending on the compound used. In general, however, the amount of the component (γ) blended based on whole curable components is preferably in the range satisfying the following formula:

$$1 ≦ \begin{matrix}\text{amount of component (γ) blended} \\ \text{based on whole curable components (\% by mass)}\end{matrix} ≦ 25 + {\sum }_{n = 2}^{∞} ( ( n - 2 ) \times ( \begin{matrix}\text{\% by mass of n-functional compound} \\ \text{based on whole curable components}\end{matrix} ) )$$

In the above formula, n is an integer of not less than 2. In other words, where the whole curable components consist only of a bifunctional compound and the component (γ), the right hand side of the above formula is 25+(2−2)×(% by mass of bifunctional compound based on whole curable components)=25.

On the other hand, where whole curable components consist of a trifunctional compound, a bifunctional compound and the component (γ), the right hand side of the above formula is 25+(2−2)×(% by mass of bifunctional compound based on whole curable components)+(3−2)×(% by mass of trifunctional compound based on whole curable components)=25+(% by mass of trifunctional compound based on whole curable components).

The amount of the component (γ) blended is preferably from 1 to 25% by mass, more preferably from 2 to 20% by mass, still more preferably from 3 to 15% by mass, based on whole curable components.

If the amount of the component (γ) blended in the composition of the present invention based on whole curable components exceeds the right hand side of the above formula:

$$25 + {\sum }_{n = 2}^{∞} ( ( n - 2 ) \times ( \% by mass of n \text{-} functional compound based on whole curable components ) ) ,$$

the heat resistance of the cured material is disadvantageously reduced.

If the amount of the component (γ) blended in the composition of the present invention based on whole curable components is less than 1% by mass, the effect of reducing the viscosity of the cured material, increasing the refractive index of the cured material and elevating the impact resistance of the cured material, which is the object of the component (γ), cannot be achieved.

The term “n-functional compound” as used herein means a compound having n radical polymerizable functional groups within one molecule.

Production Process of Component (α):

The production process of the component (α) as an essential component of the composition for plastic lenses of the present invention is described below.

The component (α) as an essential component of the composition for plastic lenses of the present invention can be produced, for example, by the following method.

Using at least one compound represented by formula (18) at a constant ratio, this compound is transesterified with one or more compound containing, as an essential component, at least one compound having an aromatic ring and two or more hydroxyl groups in the presence of a catalyst, through which step the objective compound can be obtained. Of course, the present invention is not limited thereto and other steps such as purification may be provided, if desired.

The catalyst for use in the above-described step is not particularly limited as long as the catalyst can be used for the transesterification in general. An organic metal compound is particularly preferred and specific examples thereof include tetraisopropyl titanate, tetra-n-butyl titanate, dibutyltin oxide, dioctyltin oxide, hafnium acetylacetonate and zirconium acetylacetonate, however, the present invention is not limited thereto. Among these, dibutyltin oxide and dioctyltin oxide are preferred.

The reaction temperature in this step is not particularly limited but is preferably from 100 to 230° C., more preferably 120 to 220° C. In particular, in the case of using a solvent, the reaction temperature is sometimes limited by the boiling point of the solvent.

In this step, a solvent is usually not used. However, a solvent may be used, if desired. The solvent which can be used is not particularly limited as long as it does not inhibit the transesterification. Specific examples thereof include benzene, toluene, xylene and cyclohexane, but the present invention is not limited thereto. Among these, benzene and toluene are preferred. However, as described above, the step may be performed without using a solvent.

Curable Component other than Component (α), Component (γ) and Component (γ):

For the purpose mainly of adjusting the viscosity of the composition, one or more compounds copolymerizable with the component (α), (β) or (γ) may be added to the composition for plastic lenses of the present invention, as long as they do not cause reduction in the physical properties such as reduction of the heat resistance or refractive index of the plastic lens of the present invention (IV).

Examples of this compound include monomers having a (meth)acryl group, a vinyl group or a (meth)allyl group. Specific examples thereof include methyl (meth)acrylate, isobornyl (meth)acrylate, vinyl acetate, vinyl benzoate, dibutyl maleate and dimethoxyethyl maleate.

The “(meth)acryl” as used herein includes acryl and methacryl and the “(meth)acrylate” includes acrylate and methacrylate.

Examples of the monomer having a (meth)allyl group include (meth)allyl benzoate, di(meth)allyl 1,4-cyclo-hexanedicarboxylate, di(meth)allyl 1,3-cyclohexane-dicarboxylate, di(meth)allyl 1,2-cyclohexanedicarboxylate, di(meth)allyl 4-cyclohexene-1,2-dicarboxylate, di(meth)allyl 1-cyclohexene-1,2-dicarboxylate, di(meth)allyl 3-methyl-1,2-cyclohexanedicarboxylate, di(meth)allyl 4-methyl-1,2-cyclohexanedicarboxylate, di(meth)allyl endate, di(meth)allyl chlorendate, di(meth)allyl 3,6-methylene-1,2-cyclohexanedicarboxylate, di(meth)allyl terephthalate, di(meth)allyl isophthalate and di(meth)allyl phthalate. In addition, diethylene glycol bis((meth)allyl carbonate) resin represented by CR-39 (trade name, produced by PPG) may also be used. Needless to say, the present invention is not limited to these specific examples and other monomers and the like may be used within the range of not impairing the physical properties of the plastic lens obtained by curing the composition.

The composition for plastic lenses of the present invention (III) is described below. The present invention (III) has the composition for plastic lenses of the present invention (I) or (II), wherein at least one radical polymerization initiator is contained in an amount of 0.1 to 10 parts by mass per 100 parts by mass of whole curable components in the composition for plastic lenses.

In the composition for plastic lenses of the present invention (III), a radical polymerization initiator can be added as a curing agent and this is preferred.

The radical polymerization initiator which can be added to the composition for plastic lens of the present invention (III) is not particularly limited and a commonly known radical polymerization initiator may be used as long as it does not adversely affect the physical values such as the optical properties of the plastic lens obtained by curing the composition.

The radical polymerization initiator for use in the present invention is, however, preferably soluble in other components present in the composition to be cured and at the same time, generates free radicals at 30 to 120° C. Specific examples of the radical polymerization initiator which can be added include diisopropylperoxy dicarbonate, dicyclohexylperoxy dicarbonate, di-n-propylperoxy dicarbonate, di-sec-butylperoxy dicarbonate and tert-butyl perbenzoate, but the present invention is not limited thereto. In view of curability, radical polymerization initiators having a structure represented by the following formula (19) are preferred:

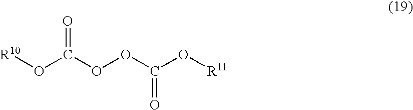

wherein R¹⁰ and R¹¹ each independently represents a group selected from the group consisting of an alkyl group having from 1 to 10 carbon atoms, a substituted alkyl group, a phenyl group and a substituted phenyl group.

Specific examples of the radical polymerization initiator represented by formula (19) include di-n-propylperoxy dicarbonate, diisopropylperoxy dicarbonate, bis(4-tert-butylcyclohexyl)peroxy dicarbonate, di-2-ethylhexylperoxy dicarbonate, di-2-ethylhexylperoxy bicarbonate, di-3-methoxybutylperoxy dicarbonate, di-sec-butylperoxy dicarbonate and di(3-methyl-3-methoxybutyl) peroxy dicarbonate. Among these, preferred are di-n-propylperoxy dicarbonate, diisopropylperoxy dicarbonate, di-2-ethoxyethylperoxy dicarbonate, di-2-ethylhexylperoxy dicarbonate and di(3-methyl-3-methoxybutyl)peroxy bicarbonate, more preferred is diisopropylperoxy dicarbonate.

The amount of the radical polymerization initiator added is from 0.1 to 10 parts by mass, preferably from 1 to 5 parts by mass, per 100 parts by mass of whole curable components contained in the composition of plastic lenses of the present invention (I) or (II). If the amount added is less than 0.1 part by mass, insufficient curing of the composition may occur. Also, addition in excess of 10 parts by mass is not preferred in view of profitability.

On considering filterability and cast working of the composition, the viscosity at 25° C. of the composition for plastic lenses of the present inventions (I) to (III) is generally 1,000 mPa·s or less, preferably 500 mPa·s or less, still more preferably 400 mPa·s or less.

The term “viscosity” as used herein is a value measured by a rotational viscometer and the details on the rotational viscometer are described in Iwanami Rikagaku Jiten, Dai 3-Pan (Iwanami Encyclopedia of Physics and Chemistry, 3rd Ed.), 3rd ed., 8th imp. (Jun. 1, 1977).

In the composition of plastic lenses of the present inventions (I) to (III), additives generally used for improving the capability of plastic lens, such as a coloring agent (e.g., dye, pigment), on ultraviolet absorbent, a light stabilizer, a mold-releasing agent and an antioxidant, may be added.

Examples of the coloring agent include organic pigments such as anthraquinone type, azo type, carbonium type, quinoline type, quinoneimine type, indigoid type and phthalocyanine type; organic dyes such as azoic dye and sulfur dye; and inorganic pigments such as titanium yellow, yellow iron oxide, zinc yellow, chrome orange, molybdenum red, cobalt violet, cobalt blue, cobalt green, chromium oxide, titanium oxide, zinc sulfide and carbon black. Needless to say, however, the present invention is not limited to these specific examples.

Examples of the mold-releasing agent include stearic acid, butyl stearate, zinc stearate, stearic acid amide, fluorine-containing compounds and silicone compounds. However, the present invention is of course not limited to these specific examples.

The ultraviolet absorbent and the light stabilizer are not particularly limited as long as it is blended in the composition but specific examples thereof include the compounds shown below. However, the present invention is of course not limited to these specific examples.

The term “ultraviolet absorbent” as used herein means a material which absorbs light energy of sunlight or fluorescent light and converts it into heat energy or the like. The term “light stabilizer” as used herein means a material which traps radicals generated due to photooxidation deterioration.

Specific examples of the ultraviolet absorbent include the compounds having a benzotriazole structure unit shown in the following structural formulae.

Examples of the compound having a benzotriazole structure unit include the compounds represented by the following structural formulae (20) to (35):

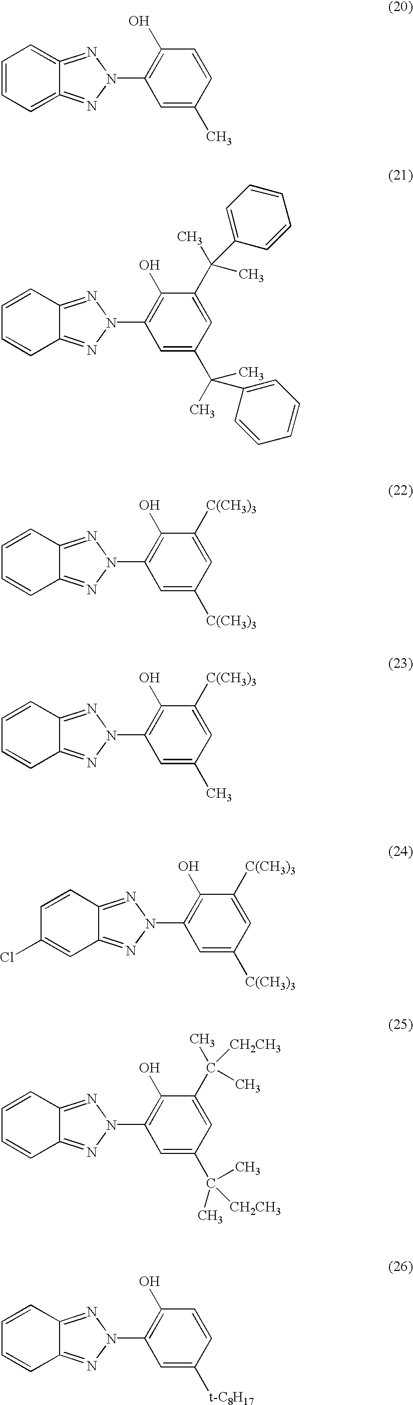

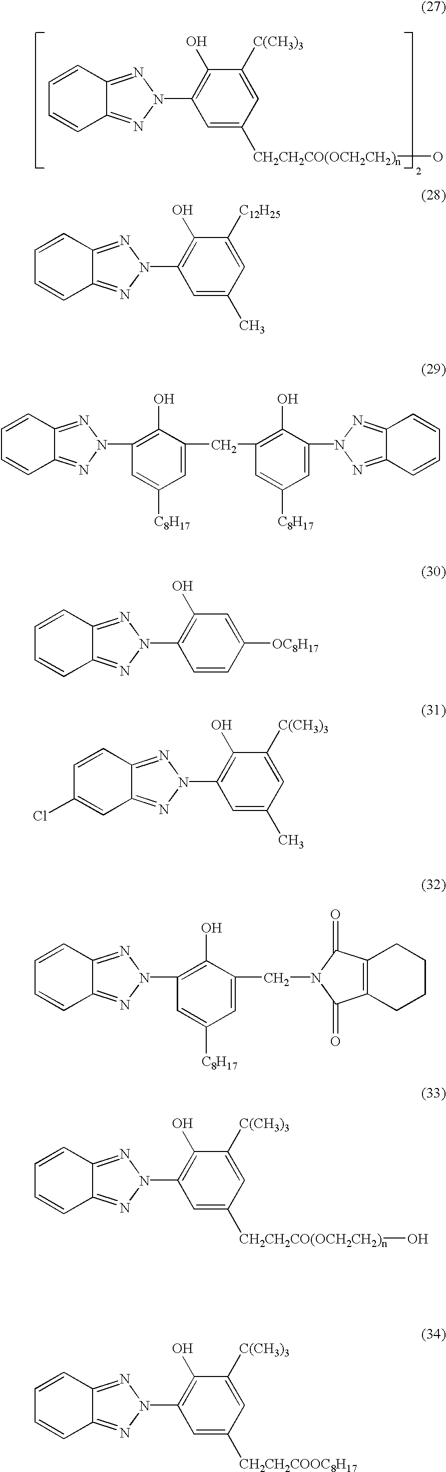

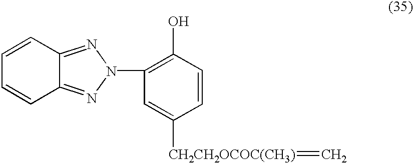

Specific examples of the benzophenone-based ultraviolet absorbent include the compounds represented by the following structural formulae (36) to (40):

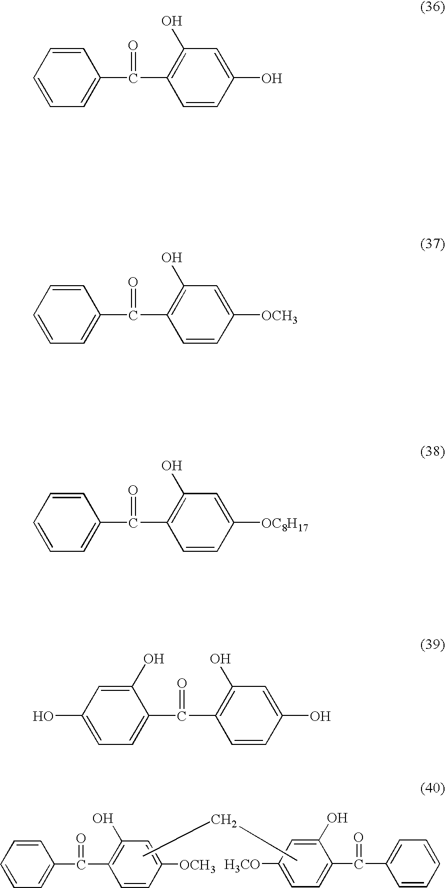

In addition, triazine-based ultraviolet absorbents represented by the following structural formula (41) and oxanilide-based ultraviolet absorbents represented by the following structural formula (42) may also be used.

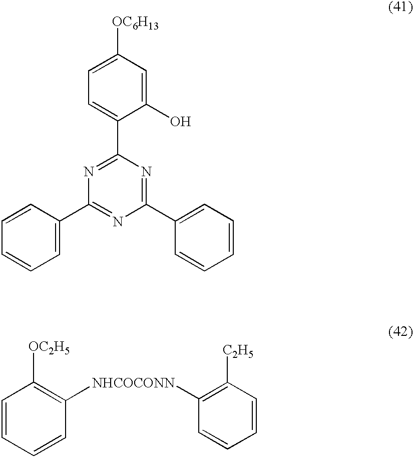

Specific examples of the light stabilizer include hindered amine-based light stabilizers (hereinafter simply referred to as “HALS”) represented by the following structural formulae (43) to (50), (52) and (54) to (57):

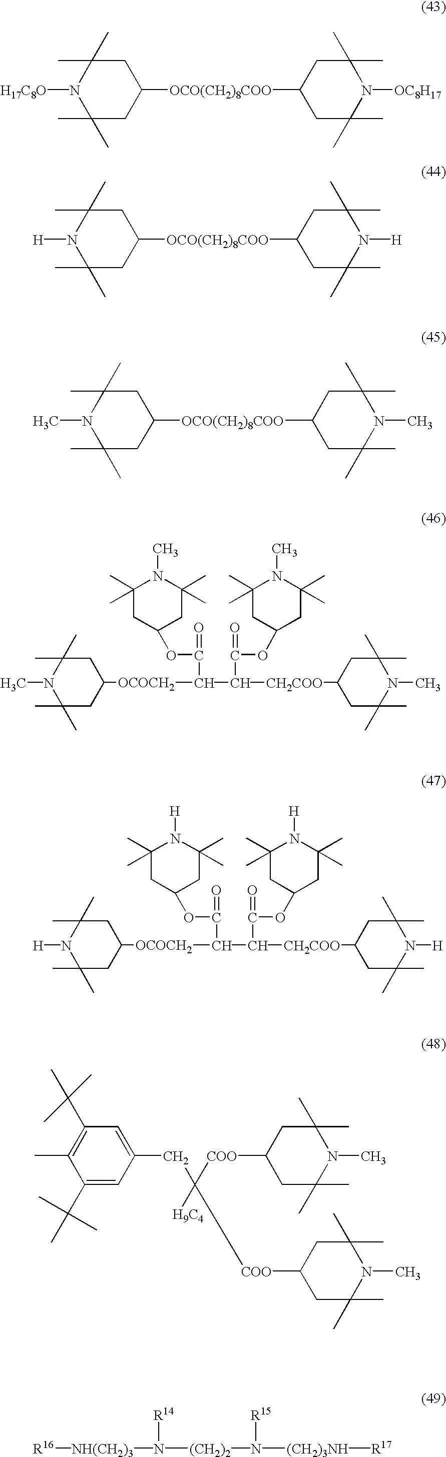

wherein R ¹⁴, R¹⁵, R¹⁶ and R¹⁷ each represents —H or

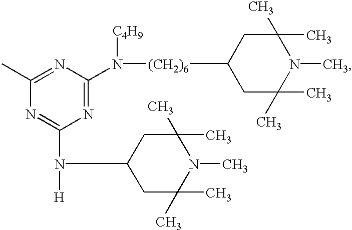

provided that the case where R¹⁴, R¹⁵, R¹⁶ and R¹⁷ all are a hydrogen atom is excluded.

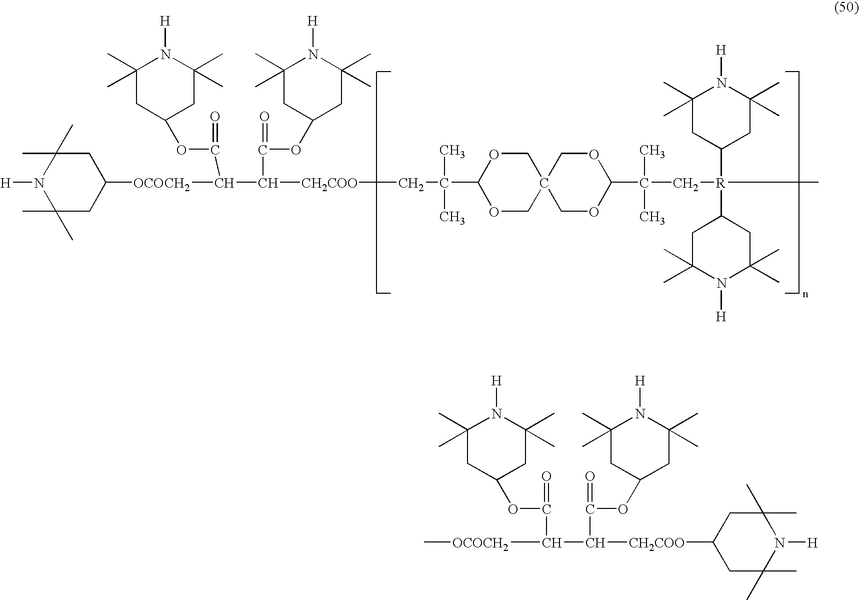

wherein R is an organic residue represented by the following structural formula (51):

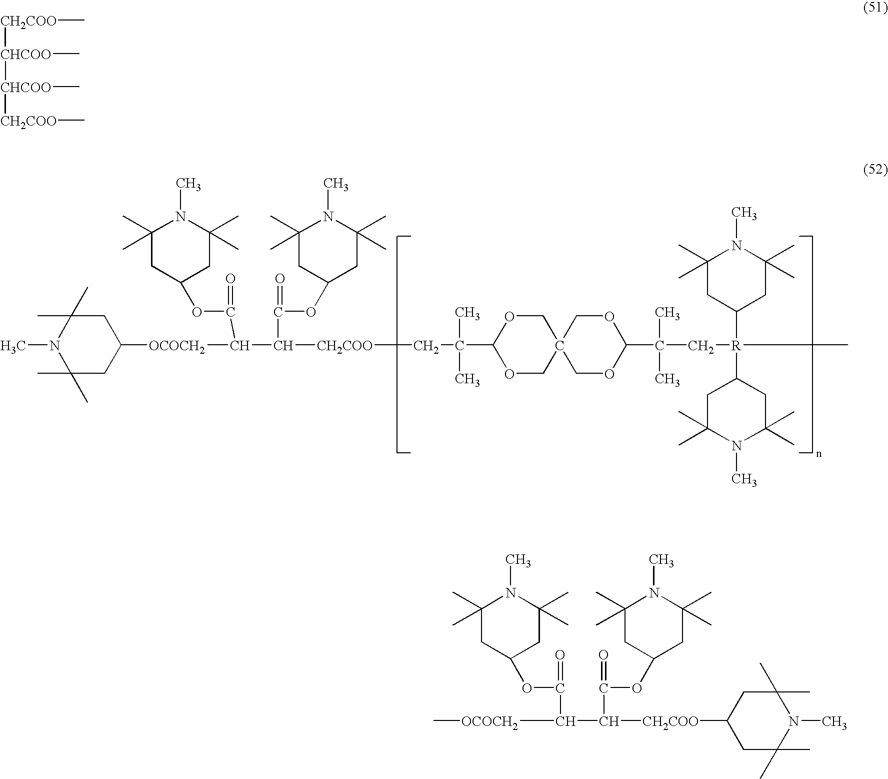

wherein R is an organic residue represented by the following structural formula (53):

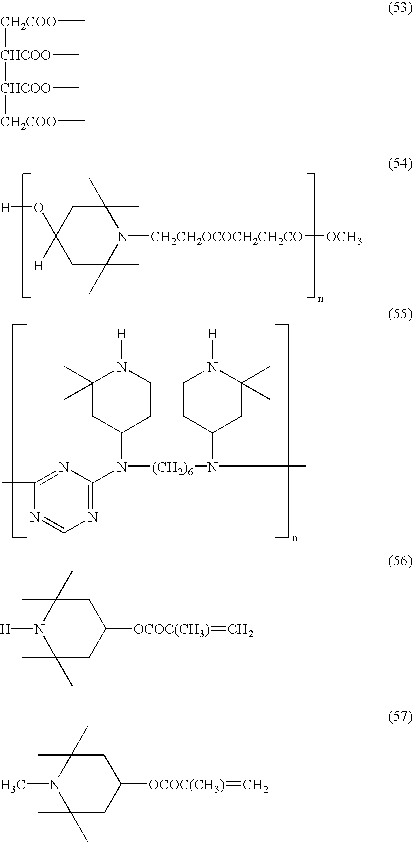

The ultraviolet absorbent and the light stabilizer may be used in combination. The ultraviolet absorbent or light stabilizer is preferably used in an amount of 0.001 to 2% by mass, more preferably from 0.05 to 1.5% by mass, base on whole curable components. If the amount added is less than 0.001% by mass, the effect of preventing deterioration cannot be fully realized and also, use in excess of 2% by mass is not preferred in view of coloration at curing or profitability.

Examples of the antioxidant which can be used include a general phenol-based antioxidant, a phosphite-based antioxidant and a thioether-based antioxidant.

Specific examples of the phenol-based antioxidant include the following compounds:

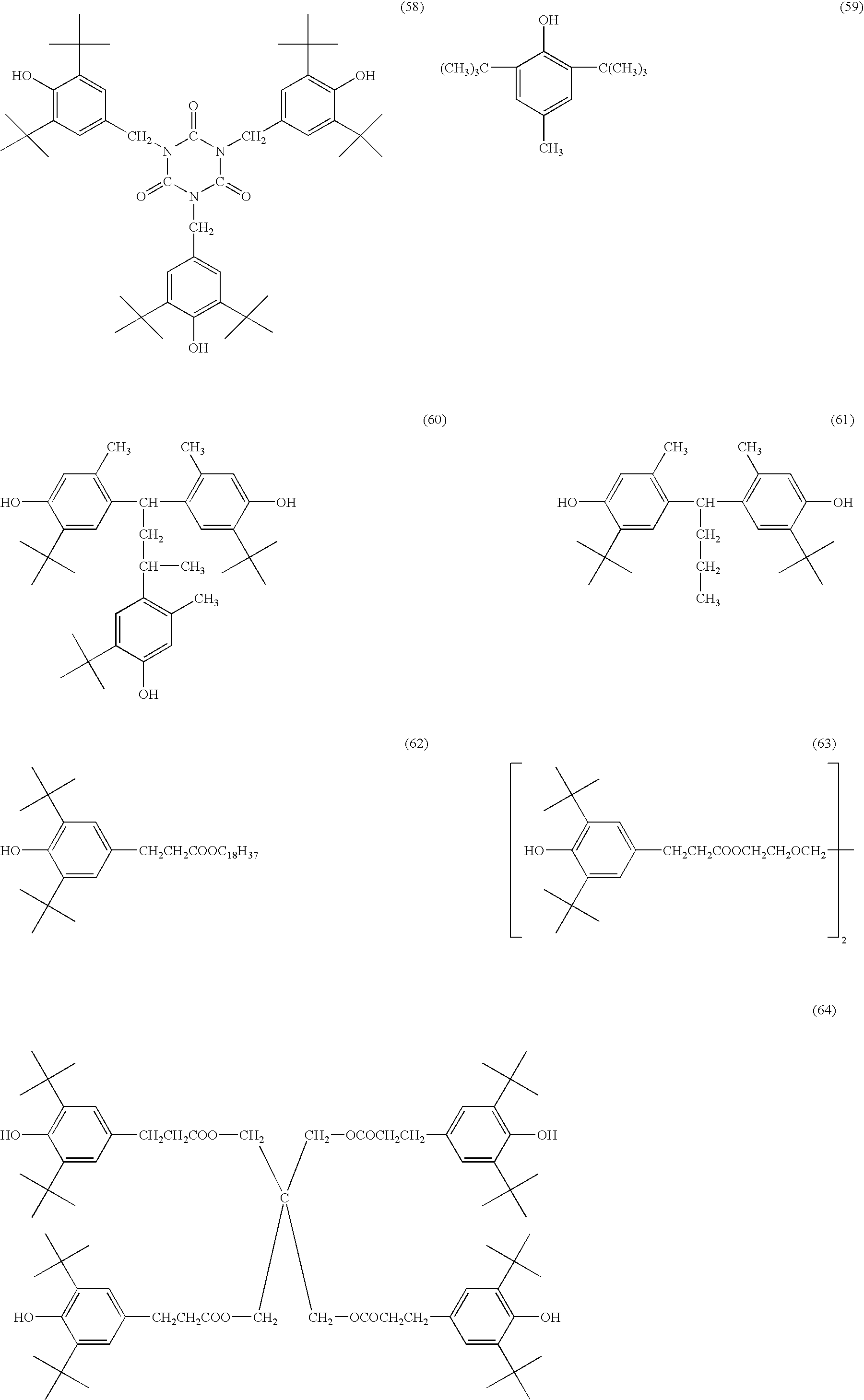

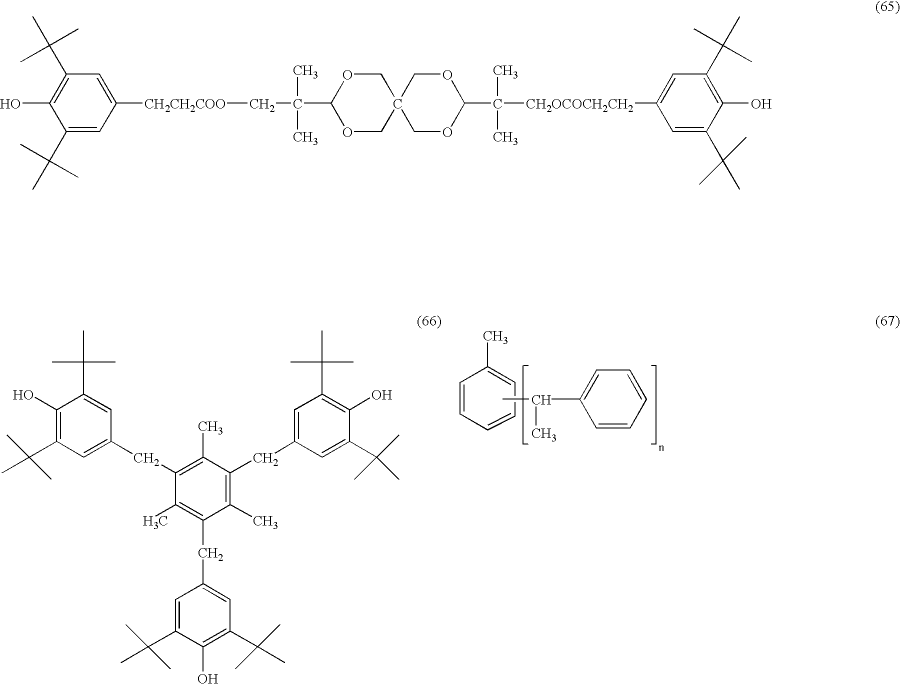

Specific examples of the phosphite-based antioxidant include the following compounds.

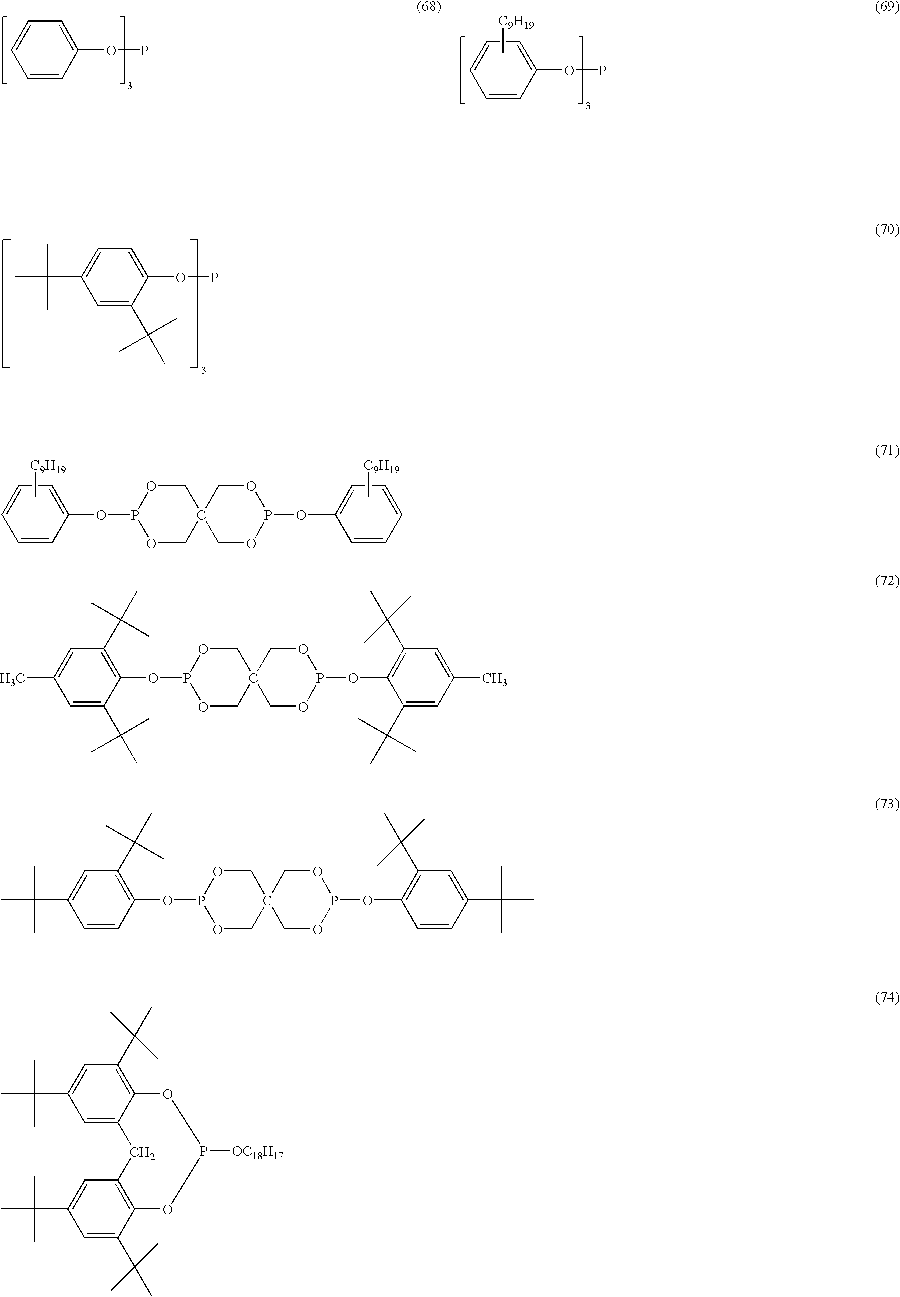

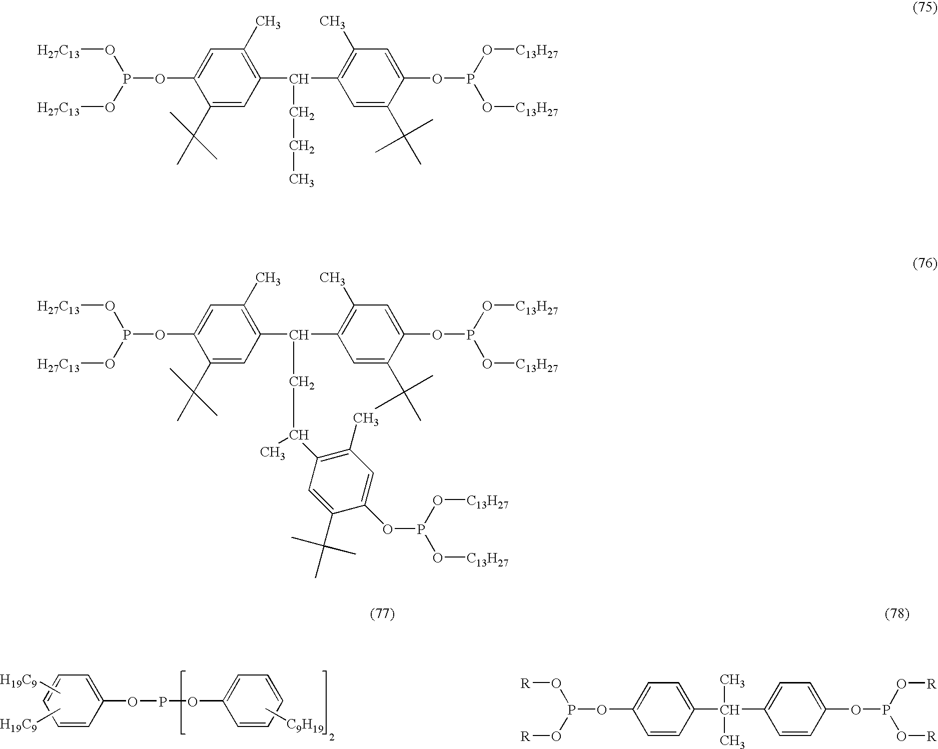

wherein R is a C₁₂-C₁₅ alkyl group.

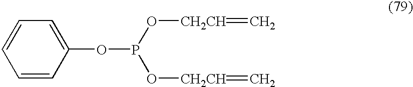

Specific examples of the thioether-based antioxidant include the following compounds.

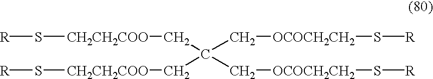

wherein R is a C₁₂-C₁₅alkyl group.

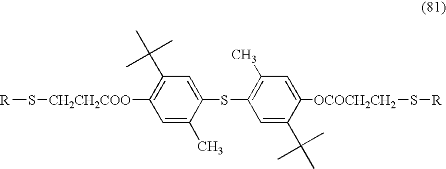

wherein R is a C₁₂ to C₁₅ alkyl group.

H₂₅C₁₂—OCOCH₂CH₂—S—CH₂CH₂COO—C₁₂H₂₅ (82)

H₂₇C₁₃—OCOCH₂CH₂—S—CH₂CH₂COO—C₁₃H₂₇ (83)

H₂₉C₁₄—OCOCH₂CH₂—S—CH₂CH₂COO—C₁₄H₂₉ (84)

H₃₇C₁₈—OCOCH₂CH₂—S—CH₂CH₂COO—C₁₈H₃₇ (85)

The above-described antioxidant may be used in combination with the ultraviolet absorbent or light stabilizer.

The amount of the antioxidant used is preferably from 0.01 to 5% by mass, more preferably from 0.1 to 3% by mass, based on whole curable components. If the amount added is less than 0.01% by mass, the effect of preventing the deterioration cannot be fully realized and also, use in excess of 5% by mass is disadvantageous in view of profitability.

In the composition for plastic lenses of the present invention, a fluorescent brightening agent such as 2,5-bis[5-tert-butylbenzoxazolyl(2)]thiophene (compound of the following structural formula (86)) may be added.

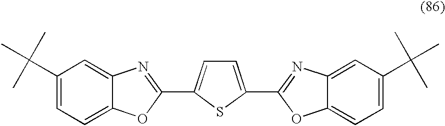

The present inventions (IV) and (V) will now be described below. The present invention (IV) is a plastic lens obtained by curing the composition for plastic lenses described in any one of the present inventions (I) to (III).

The present invention (V) is a process for producing a plastic lens of the present invention (IV).

In the present invention, the mold-processing of the composition for plastic lenses is suitably cast molding. More specifically, a molding method of adding a radical polymerization initiator to the composition, filling the composition into a mold fixed by an elastomer gasket or spacer through a line, and heat-curing it in an oven may be used.

The construction material used as a mold here is metal or glass. In general, the mold for plastic lenses must be cleaned after the cast-molding and such cleaning is usually performed using a strong alkali solution or a strong acid. Unlike metal, glass is virtually unchanged in terms of quality by the cleaning and can be easily polished and thereby its surface roughness greatly reduced. Because of these reasons, glass is preferably used.

The curing temperature at the time of molding the composition for plastic lenses described in any one of the present inventions (I) to (III) is from about 30 to 120° C., preferably from 40 to 100° C. Taking into account shrinkage or strain at the time of curing, the curing temperature is preferably facilitated by way of a method which allows the curing to proceed gradually while raising the temperature. The curing time is generally from 0.5 to 100 hours, preferably from 3 to 50 hours, more preferably from 10 to 30 hours.

The plastic lens of the present invention can be dyed similarly to normal plastic lenses.

The method for dyeing the plastic lens of the present invention is not particularly limited and any method may be used as long as it is a known dyeing method for plastic lenses. Among these, a dip dyeing method conventionally known as a general method is preferred. The term “dip dyeing method” as used herein means a method of dispersing a disperse dye together with a surfactant in water to prepare a dyeing solution and dipping a plastic lens in this dyeing solution under heating, thereby dyeing the plastic lens.

The method for dyeing the plastic lens is not limited to this dip dyeing method but other known methods may be used, for example, a method of sublimating an organic pigment and thereby dyeing a plastic lens (see, JP-B-35-1384 (the term “JP-B” as used herein means “examined Japanese patent publication”)) or a method of sublimating a sublimable dye and thereby dyeing a plastic lens (see, JP-B-56-159376 and JP-B-1-277814) may be used. In view of simplicity of operation, the dip dyeing method is most preferred.

The present invention is further illustrated below with reference to examples. However, the present invention should not be construed as being limited thereto.

Various physical properties were measured as follows.

1. Refractive Index (n_{D}) and Abbe Number

A test piece of 9 mm×16 mm×4 mm was prepared and measured in terms of refractive index (n_{D}) and Abbe number (ν_{D}) at 25° C. using “Abbe Refractometer 1T” manufactured by Adaco. The contact solvent used was α-bromonaphthalene.

2. Viscosity

In Examples 1 to 5 and Comparative Example 1 described later, 5.2 ml of a sample was charged into a specified vessel and the viscosity was measured at a measurement temperature of 25° C. by a B-Type Viscometer (Model B8U) manufactured by Tokyo Keiki Co., Ltd. using an HH-1 rotor at a rotation number of 100 rpm.

3. Barcol Hardness

The Barcol hardness was measured using Model 934-1 according to JIS K 6911.

4. Specific Gravity of Cured Material

The specific gravity of the cured material after the curing was measured by the sink-float method (at 23° C.) described in JIS K 7112.

### PRODUCTION EXAMPLE 1

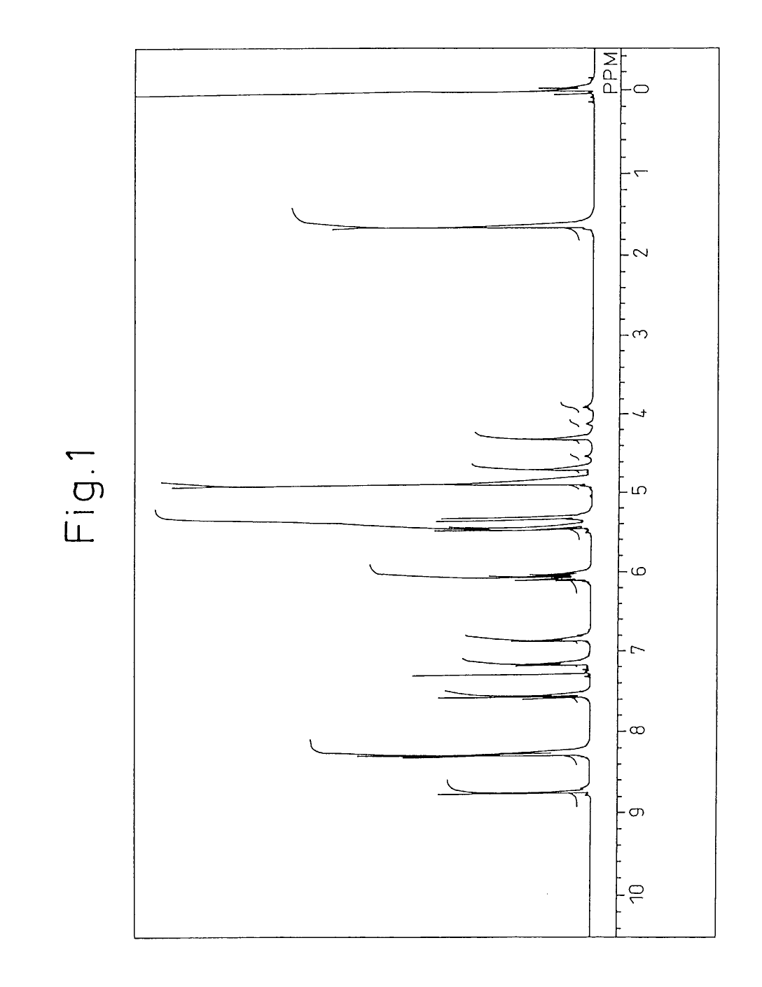

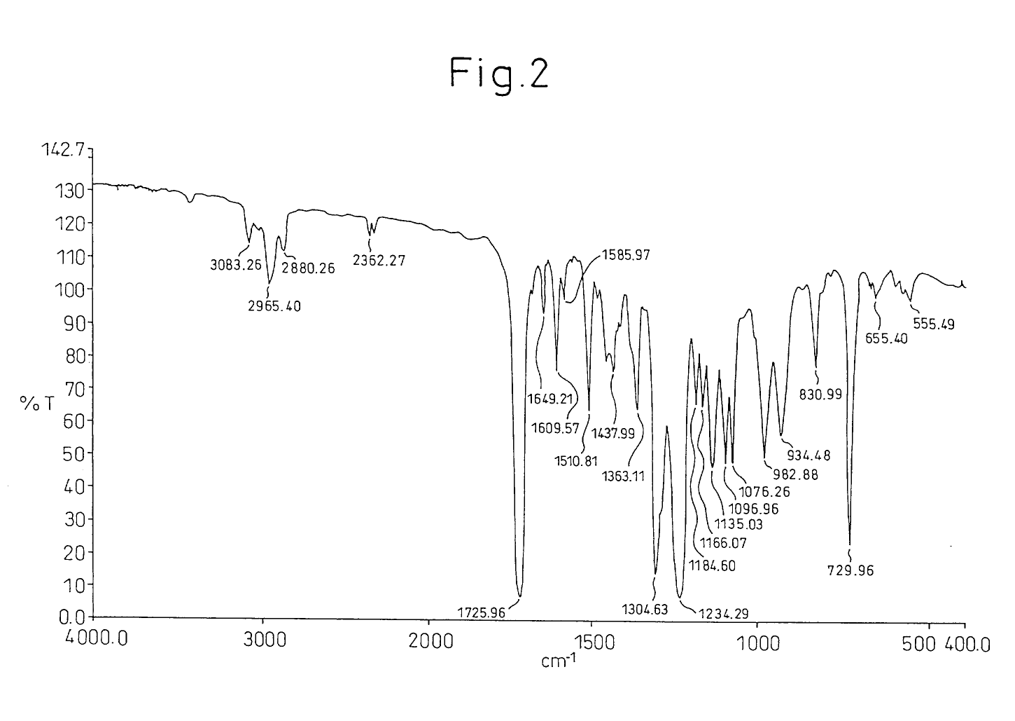

Into a 3 L three-neck flask with a distillation unit, 1,108.2 g (4.5 mol) of diallyl isophthalate, 316.4 g (1.0 mol) of 2 mol ethylene oxide adduct of bisphenol A (Newcol 1900: trade name, produced by Nihon Nyukazai K.K.) and 1.11 g (0.1 wt % (based on diallyl isophthalate)) of dibutyltin oxide were charged. The system was heated at 180° C. in a nitrogen stream to distill off allyl alcohol generated. When about 81 g of allyl alcohol was distilled off, the pressure inside the reaction system was reduced to 1.33 kPa to accelerate the distillation of allyl alcohol. After a theoretical amount (116.2 g) of allyl alcohol was distilled off, the system was heated for another one hour and then kept at 190° C. and 0.13 kPa for one hour. Thereafter, the reactor was cooled, as a result, 1,308.4 g of an allyl ester compound was obtained (hereinafter referred to as “Sample A”). FIG. 1 and FIG. 2 show 400 MHz ¹H-NMR spectrum (solvent: CDCl₃) and FT-IR spectrum of Sample A, respectively.

Sample A was analyzed by gas chromatography (GC-14B manufactured by Shimadzu Corporation, hydrogen flame ionization detector, column used: OV-17 of 0.5 m, column temperature: constant at 130° C. for 4 minutes and after raising to 160° C. at 32° C./min, constant at 160° C.) and found to contain 55% by mass of diallyl isophthalate.

### PRODUCTION EXAMPLE 2

In the same manner as in Production Example 1 except for using 1,231.3 g (5.0 mol) of diallyl isophthalate in place of 1,108.2 g (4.5 mol) of diallyl isophthalate, 1,431.5 g of an allyl ester compound was obtained (hereinafter referred to as “Sample B”).

Sample B was analyzed by gas chromatography (GC-14B manufactured by Shimadzu Corporation, hydrogen flame ionization detector, column used: OV-17 of 0.5 m, column temperature: constant at 130° C. for 4 minutes and after raising to 160° C. at 32° C./min, constant at 160° C.) and found to contain 60% by mass of diallyl isophthalate.

### PRODUCTION EXAMPLE 3

In the same manner as in Production Example 1 except for using 288.3 g (1.0 mol) of 2 mol ethylene oxide adduct of bisphenol F in place of 316.4 g (1.0 mol) of 2 mol ethylene oxide adduct of bisphenol A, 1,280.4 g of an allyl ester compound was obtained (hereinafter referred to as “Sample C”).

Sample C was analyzed by gas chromatography (GC-14B manufactured by Shimadzu Corporation, hydrogen flame ionization detector, column used: OV-17 of 0.5 m, column temperature: constant at 130° C. for 4 minutes and after raising to 160° C. at 32° C./min, constant at 160° C.) and found to contain 59% by mass of diallyl isophthalate.

### PRODUCTION EXAMPLE 4

Into a 2 L three-neck flask with a distillation unit, 1,108.2 g (4.5 mol) of diallyl isophthalate, 216.3 g (0.75 mol) of 2 mol ethylene oxide adduct of bisphenol F, 103.6 g of p-xylylene glycol (0.75 mol) and 1.11 g (0.1 wt % (based on diallyl isophthalate)) of dibutyltin oxide were charged. The system was heated at 180° C. in a nitrogen stream to distill off allyl alcohol generated. When about 120 g of allyl alcohol was distilled off, the pressure inside the reaction system was reduced to 1.33 kPa to accelerate the distillation of allyl alcohol. After a theoretical amount (174.2 g) of allyl alcohol was distilled off, the system was heated for another one hour and then kept at 190° C. and 0.13 kPa for one hour. Thereafter, the reactor was cooled, as a result, 1,224.8 g of an allyl ester compound was obtained (hereinafter referred to as “Sample D”).

Sample D was analyzed by gas chromatography (GC-14B manufactured by Shimadzu Corporation, hydrogen flame ionization detector, column used: OV-17 of 0.5 m, column temperature: constant at 130° C. for 4 minutes and after elevating to 160° C. at 32° C./min, constant at 160° C.) and found to contain 50% by mass of diallyl isophthalate.

### EXAMPLE 1

As shown in Table 1, 83.0 parts by mass of the allyl ester compound as Sample B, 8.0 parts by mass of diallyl biphenyl-2,2′-dicarboxylate, 9 parts by mass of p-phenylbenzoic acid and 3 parts by mass of diisopropylperoxy dicarbonate (IPP) were blended and mixed with stirring to give a completely homogeneous solution composition. The viscosity at this time was measured. Thereafter, a vessel containing this solution was placed in a desiccator capable of depressurization and the pressure was reduced by a vacuum pump for about 15 minutes to degas the solution. The resulting solution composition was injected by a syringe into a mold fabricated from a glass-made mold for ophthalmic plastic lenses and a resin-made gasket, while taking care to prevent intermixing of gas, and then cured in an oven according to a temperature-rising program of 40° C. for 7 hours, 40 to 60° C. for 10 hours, 60 to 80° C. for 3 hours, 80° C. for 1 hour, and 85° C. for 2 hours.

The lens obtained was measured in terms of refractive index, Abbe number, Barcol hardness and specific gravity at 23° C. The results are shown in Table 1.

TABLE 1

| | | | Example | Example | Example | Example | Example | Comparative |
|---|---|---|---|---|---|---|---|---|
| | | | 1 | 2 | 3 | 4 | 5 | Example 1 |
| Blending | Sample A | Diallyl isophthalate | | | | 39 | | 38.5 |
| (parts by | | Compound of structural | | | | 32 | | 31.5 |
| mass) | | formula (87) | | | | | | |
| | Sample B | diallyl terephthalate | 50 | | | | | |
| | | Compound of structural | 33 | | | | | |
| | | formula (87) | | | | | | |
| | Sample C | diallyl terephthalate | | 49 | | | 49 | |
| | | Compound of structural | | 34 | | | 34 | |
| | | formula (88) | | | | | | |
| | Sample D | diallyl terephthalate | | | 41.5 | | | |
| | | Compound of structural | | | 41.5 | | | |
| | | formula (89) | | | | | | |

| | Diallyl biphenyl-2,2′-dicarboxylate | 8 | 8 | 8 | 20 | 8 | |
|---|---|---|---|---|---|---|---|
| | CR-39 | 15 | | | | | |
| | Allyl p-phenylbenzoate | 9 | | | 9 | 9 | |
| | Allyl β-naphthoate | | 9 | 9 | | | |
| | Allyl benzoate | | | | | | 15 |
| Viscosity (25° C.) (mPa · s) | | 330 | 260 | 300 | 270 | 260 | 200 |
| Initiator IPP (parts by mass) | | 3 | 3 | 3 | 3 | 3 | 3 |
| Physical | Refractive index, n_{D} | 1.589 | 1.595 | 1.591 | 1.596 | 1.594 | 1.545 |
| properties | Abbe number | 31.2 | 30.0 | 32.0 | 29.9 | 30.6 | 40.0 |
| of cured | Barcol hardness | 45 | 33 | 35 | 47 | 38 | 35 |
| material | Specific gravity | 1.25 | 1.26 | 1.26 | 1.26 | 1.26 | 1.28 |

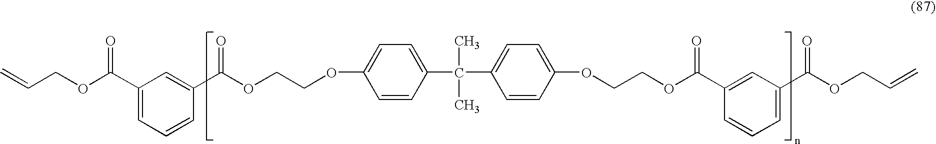

wherein n is an integer of 1 or more.

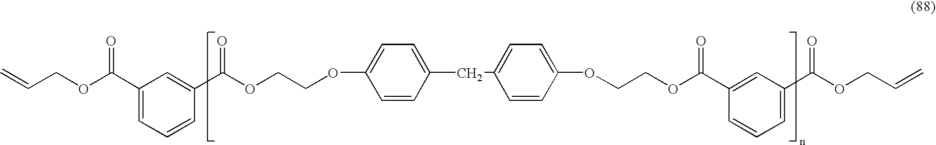

wherein n is an integer of 1 or more.

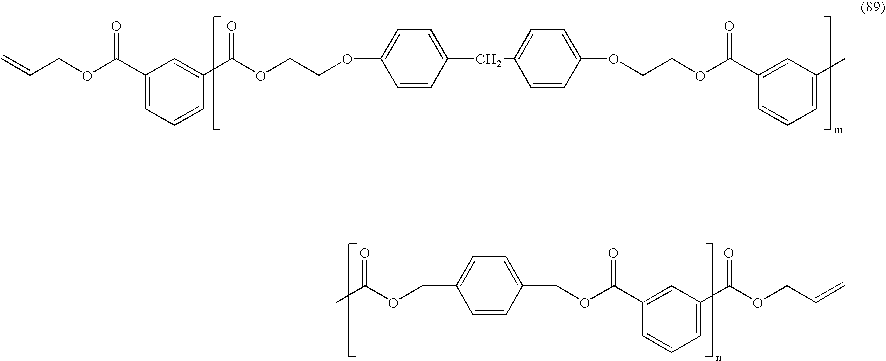

wherein m and n are each independently 0 or an integer of 1 or more and (m+n) is an integer of 1 or more.

### EXAMPLES 2 TO 5 AND COMPARATIVE EXAMPLES 1

Compositions were prepared according to the blending shown in Table 1 and, in the same manner as in Example 1, measured in terms of viscosity and then cured. The lenses obtained were measured in terms of refractive index, Abbe number, Barcol hardness and specific gravity at 23° C. The results are shown in Table 1.

### Industrial Applicability

It is proved that according to the present invention, a composition for plastic lens having a viscosity suitable for application to plastic lens materials and other optical materials and capable of providing a cured material having a relatively high refractive index and a small specific gravity can be provided. Also, a plastic lens obtained by curing the composition is provided.

1. A composition for plastic lenses, comprising the following component (α) and component (β) as essential components:
Component (α):
at least one compound selected from the compounds having at least one group represented by the following formula (1) as a terminal group and having a group represented by the following formula (2) as a repeating unit; 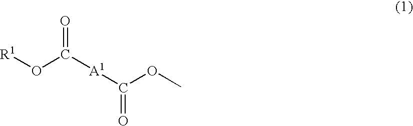
wherein each R¹ independently represents an allyl group or a methallyl group and each A¹ indepedently represents an organic residue derived from a divalent carboxylic acid or carboxylic anhydride; 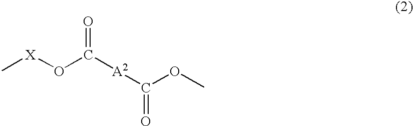
wherein each A² independently represents an organic residue derived from a divalent carboxylic acid or carboxylic anhydride and each X indepedently represents an organic residue and the X's represent one or more organic residues essentially containing an organic residue derived from a compound having an aromatic ring and two or more hydroxyl groups, provided that by the ester bonding, X can have a branched structure having a group represented by formula (1) as a terminal group and a group represented by formula (2) as a repeating unit;
Component (β):
at least one compound selected from the compounds represented by the following formula (3): 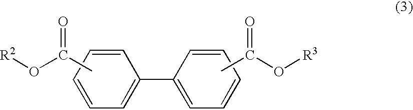
wherein R² and R³ each independently represents an allyl group or a methallyl group.

2. A composition for plastic lenses according to claim 1, wherein the component (α) is contained in an amount of 10 to 95% by mass and the component (β) is contained in an amount of 5 to 90% by mass.

3. A composition for plastic lenses, comprising the following component (α), component (β) and component (γ) as essential components:
Component (α):
at least one compound selected from the compounds having at least one group represented by the following formula (1) as a terminal group and having a group represented by the following formula (2) as a repeating unit; 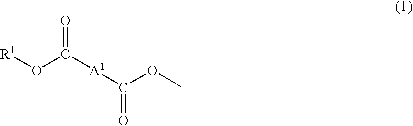
wherein each R¹ independently represents an allyl group or a methallyl group and each A¹ indepedently represents an organic residue derived from a divalent carboxylic acid or carboxylic anhydride; 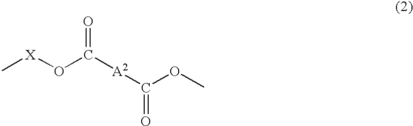
wherein each A² independently represents an organic residue derived from a divalent carboxylic acid or carboxylic anhydride and each X indepedently represents an organic residue and the X's represent one or more organic residues essentially containing an organic residue derived from a compound having an aromatic ring and two or more hydroxyl groups, provided that by the ester bonding, X can have a branched structure having a group represented by formula (1) as a terminal group and a group represented by formula (2) as a repeating unit;
Component (β):
at least one compound selected from the compounds represented by the following formula (3): 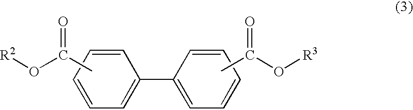
wherein R² and R³ each independently represents an allyl group or a methallyl group;
Component (γ):
at least one monofunctional compound selected from the group consisting of monofunctional compounds having two or more benzene rings within one molecule, monofunctional compounds having a naphthalene ring within one molecule and monofunctional compounds having a benzene ring and a halogen atom within one molecule.

4. A composition for plastic lenses according to claim 3, wherein the component (α) is contained in an amount of 10 to 70% by mass, the component (β) is contained in an amount of 5 to 90% by mass and the component (γ) is contained in an amount of 1 to 25% by mass.

5. A composition for plastic lenses according to any one of claims 1 to 4, wherein the compound having an aromatic ring and two or more hydroxyl groups is selected from the compounds represented by the following formulae (7) to (9): 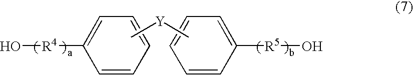
wherein each R⁴ independently represents an organic group selected from the organic groups represented by the following structural formulae (10) to (12), each R⁵ independently represents an organic group selected from the organic groups represented by the following structural formulae (13) to (15), a and b are each independently 0 or an integer of 1 to 10, and Y represents an organic group represented by the following structural formulae (16) or (17); 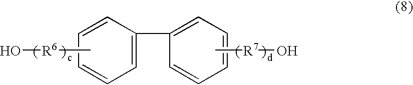
wherein each R⁶ independently represents an organic group selected from the organic groups represented by structural formulae (10) to (12), each R⁷ independently represents an organic group selected from the organic groups represented by structural formulae (13) to (15), and c and d are each independently 0 or an integer of 1 to 10; 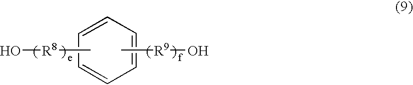
wherein each R⁸ independently represents a methylene group or an organic group selected from the organic groups represented by structural formulae (10) to (12), each R⁹ represents an organic group selected from the organic groups represented by structural formulae (13) to (15), and e and f are each independently 0 or an integer of 1 to 10.
—CH₂CH₂O— (10) 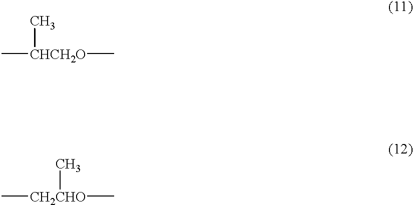
—OCH₂CH₂— (13) 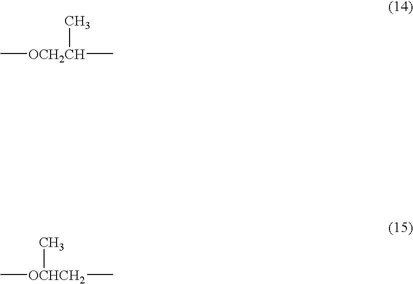
—CH₂— (16)

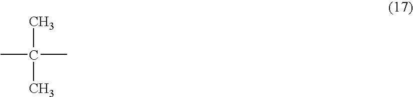

6. A composition for plastic lenses according to any one of claims 1 to 5, wherein at least one radical polymerization initiator is contained in an amount of 0.1 to 10 parts by mass per 100 parts by mass of whole curable components in the composition for plastic lenses.

7. A composition for plastic lenses according to claim 6, wherein the radical polymerization initiator is selected from the compounds represented by the following formula (19). 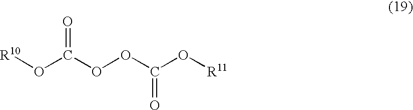
wherein R¹⁰ and R¹¹ each independently represents a group selected from the group consisting of an alkyl group having from 1 to 10 carbon atoms, a substituted alkyl group, a phenyl group and a substituted phenyl group.

8. A composition for plastic lenses according to any one of claims 1 to 7, which has a viscosity at 25° C. of not more than 500 mPa·s.

9. A plastic lens obtained by curing a composition for plastic lenses as set forth in any one of claims 1 to 8.

10. A process for producing a plastic lens, comprising curing a composition for plastic lenses as set forth in any one of claims 1 to 8.

11. A process according to claim 10, wherein the composition for plastic lenses is cured by casting polymerization at a curing temperature of 30 to 120° C. for a curing time of 0.5 to 100 hours.
